(19) United States
(12) Patent Application Publication
(10) Pub. No.: US 2026/0021050 A1
(43) Pub. Date: Jan. 22, 2026

(54) POLY(AMINO ACID) BASED CAPSULES

(71) Applicant: Agfa-Gevaert NV, Mortsel (BE)

(72) Inventor: Johan Loccufier, Mortsel (BE)

(73) Assignee: Agfa-Gevaert NV, Mortsel (BE)

(21) Appl. No.: 19/099,961

(22) PCT Filed: Jul. 31, 2023
(86) PCT No.: PCT/EP2023/071133

§ 371 Date: Jan. 30, 2025

(30) Foreign Application Priority Data
Aug. 3, 2022 (EP) 22188434.9

(51) Int. Cl. A61K 9/48; B01J 13/18

(52) U.S. Cl. CPC A61K 9/4825; B01J 13/185

(57) ABSTRACT

A capsule consisting of a polymeric shell surrounding a core, the core comprises an organic compound, the polymeric shell comprises a polypeptide comprising a moiety according to general formula I and a moiety according to general formula II General formula I General formula II The organic compound can be a marine oil, a vegetable oil, an essential oil, a fragrance, a flavour, an insect repellent, a flame retardant, an active pharmaceutical ingredient or an agrochemical.

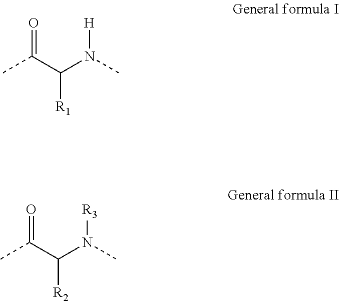

### TECHNICAL FIELD

It is an object of the invention to provide a poly(amino acid) based capsule. It is a further object of the invention to provide a synthetic method for the preparation of poly(amino acid) based capsules.

### BACKGROUND ART

Biodegradability of polymers is an ever increasing demand in a whole set of applications, especially those applications holding the risk of polymers ending up in the environment. Therefore, more and more bio-based approaches are appearing in different fields of technology. Encapsulation is a very promising technology for controlled release of different chemicals, e.g. biological active products or fragrances, for protection of hydrolytically sensitive compounds in aqueous formulations and for separating reactivity in single fluid formulations. Amongst others, life sciences, agrochemicals and cosmetics are major fields of application for encapsulation, where release of encapsulated chemistry in the environment or contact with a biological environment is unavoidable. Therefore, biodegradability and biocompatibility will become an absolute requirement in all of these applications.

Nano- and microcapsules can be prepared using both chemical and physical methods. Encapsulation methodologies include complex coacervation, liposome formation, spray drying and precipitation and polymerisation methods. For technological applications, interfacial polymerisation is a particularly preferred technology, which has been reviewed by Zhang Y. and Rochefort D. (Journal of Microencapsulation, 29(7), 636-649 (2012) and by Salaün F. (in Encapsulation Nanotechnologies, Vikas Mittal (ed.), chapter 5, 137-173 (Scrivener Publishing LLC (2013)).

Polymerization methods are particularly preferred, as they allow the highest control in designing the capsules. More preferably interfacial polymerization and most preferably interfacial polycondensation is used to prepare the capsules for technological applications. In interfacial polymerization, polymerization occurs at the interface of the oil drops in an oil-in-water emulsion or at the interface of the water drops in water-in-oil emulsions. In interfacial polycondensation, two reactants meet at the interface of the emulsion droplets and react rapidly.

In general, interfacial polymerisation requires the dispersion of an oleophilic phase in an aqueous continuous phase or vice versa. Classically each of the phases contains at least one dissolved monomer (a first shell component) that is capable of reacting with another monomer (a second shell component) dissolved in the other phase. Upon polymerisation, a polymer is formed that is insoluble in both the aqueous and the oleophilic phase. As a result, the formed polymer has a tendency to precipitate at the interface of the oleophilic and aqueous phase, hereby forming a shell around the dispersed phase, which grows upon further polymerisation.

Interfacial polymerisation technologies known in the prior art rely on the polymerisation of often petrochemical based synthetic monomers, leading to shell chemistry typically selected from polyamides, polyurea, polyurethanes, polyesters, polycarbonates or combinations thereof. Polycondensation products of aldehydes and other monomers such as melamine or urea are also well documented in the literature. However, in general all of this shell chemistry leads to non or scarcely degradable polymers.

Poly(amino acids) are a well-known class of biocompatible and biodegradable polymers and would be a preferred class of shell polymers for biocompatible micro- and nanocapsule design. However, classical interfacial polycondensation as described above, is not suited as preparation method for preparing poly(amino acid) based capsules.

Poly(amino acids) can be prepared by the polymerization of N-carboxy-anhydride monomers (NCA's) in a heterogeneous water-solvent-system. Wang et al. (Journal of Biomedical Research Part B: Applied Biomaterials, 89B(1), 45-54 (2009)) described the preparation of glycopeptide microspheres starting from acylated chitosan as initiator for graft-polymerization of NCA's in a heterogeneous water-solvent mixture. The disclosed microspheres were prepared using L-leucine as amino acid. The spheres have a particle size of several tens of microns up to a few hundred microns and did not contain specific core material.

Jacobs et al. disclosed mini-emulsion polymerization using NCA's in a heterogeneous water-solvent-mixture (J. Am. Soc., 141, 12522-12526 (2019)). The particle size was in the range of 200 nm. The particles did not contain core material. Furthermore, the used L-cysteine amino acid shows secondary structure arrangements such as β-sheet confirmations during the shell formation of the caps. These secondary structure arrangements negatively interact with the polymerisation of the NCA's leading to deformation of the particles and leading to a reduced process latitude of the capsule production on an industrial scale.

It is further observed that secondary structure arrangements of polyaminoacids show a limited biodegradability.

In a lot of approaches, amphiphilic block copolymers containing poly(amino acid) blocks are prepared separately and assembled into micelle like capsules or transferred into capsules using coacervation type of approaches. The self-assembly of amphiphilic block copolymers into micelles can hold up core material. Micelle based capsules have the disadvantage of a much weaker shell than a capsule with a polymeric shell. In many systems, a crosslinking of the shell of micellar systems is therefore required.

WO96/40279 discloses the production of microspheres via cavitation of amphiphilic poly amino acid block co-polymers. Stable microspheres can only be achieved for a certain hydrophobic-hydrophilic balance of the block co-polymers, hence limiting the number of suitable amino acid polymers considerably.

In the literature, for example, Jianxun Ding in Nanotechnology 22 (2011) 494012, different block copolymer based micelles have been documented as encapsulation technology. Micelle like capsules mostly need a liquid medium to retain its spherical structure such as to hold the core material in the inside of the micelle. Isolation of the micelle in a dried state is hence therefor very difficult or not possible. Micelle like capsules have a limited range of obtainable particle sizes in contrast to capsules obtained by interfacial polymerization, more particularly in the lower particle size range. Furthermore, the approaches by means of amphiphilic block copolymers allow good control on the polymer structure but require exhaustive synthetic procedures to prepare the well-defined polymers, making them less suitable for technical applications in contrast to interfacial polymerization based technologies.

Other encapsulation technologies such as e.g. complex coacervation, require thorough control of the operational process window, which is often quite narrow, limiting the flexibility of the technology on an industrial scale.

Therefore, there is still a need for encapsulation approaches for the design of poly(amino acid) based capsules having a process latitude fit for use in an industrial environment and wherein the shell of the capsule shows an excellent biodegradability.

### SUMMARY OF INVENTION

Now it has been found that core shell structures, having a polypeptide shell comprising a moiety according to Formula I, can realize the objects of the present invention.

The present invention comprises capsules consisting of a polymeric shell based on poly(amino acids) surrounding a core as defined in Claim 1.

According to another aspect, the present invention includes a method of preparing the capsules of Claim 1. This method is defined in Claim 14.

Other features, elements, steps, characteristics and advantages of the present invention will become more apparent from the following detailed description of preferred embodiments of the present invention. Specific embodiments of the invention are also defined in the dependent claims.

### DESCRIPTION OF EMBODIMENTS

### A. The capsule

The objects of the present invention are realized by a core shell structure, wherein said core comprises an organic compound and the shell comprises a polypeptide comprising the moiety according to general formula I and a moiety according to general formula Il

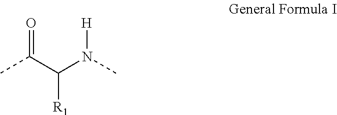

Wherein
R₁ is selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group.

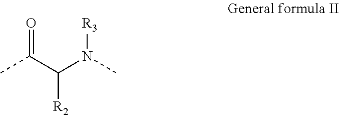

R₂ is selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₃ is selected from the group consisting of a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₂ and R₃ may represent the necessary atoms to form a five to eight membered ring.

The organic compound is preferably a low volatile substantially hydrophobic organic compound.

### A.1. The Shell

The shell, is a polymeric shell which comprises a polypeptide comprising the moiety according to general formula I and the moiety according to general formula II. Preferably, the polypetide is obtained by oligomerization or polymerization of a N-carboxy-anhydride monomer according to general structure III and a N-carboxy-anhydride monomer according to general structure IV.

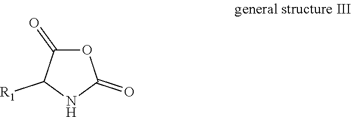

wherein
R₁ is selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group

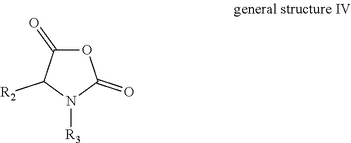

wherein
R₂ is selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₃ is selected from the group consisting of a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₂ and R₃ may represent the necessary atoms to form a five to eight membered ring.

### A.1.1. N-Carboxy-Anhydride Monomer According to General Structure III

In a preferred embodiment R₁ is selected from the group consisting of a hydrogen, a substituted or unsubstituted alkyl group, a substituted or unsubstituted aralkyl group and a substituted or unsubstituted aryl group, an unsubstituted alkyl group, an unsubstituted aralkyl group and an unsubstituted aryl group being particularly preferred.

In further preferred embodiment, the N-carboxy-anhydride monomer according to general structure III is selected from the group consisting of a glycine derivative, an alanine derivative, a leucine derivative, a phenylalanine derivative, a phenylglycine derivative, a valine derivative, a glutamic acid derivative, an aspartic acid derivative, a lysine derivative, an ornithine derivative, a histidine derivative, a methionine derivative, a cysteine derivative, an arginine derivative, a tryptophane derivative, a cysteine derivative, an isoleucine derivative, a tyrosine derivative and a serine derivative.

Both D- and L-amino acid derivatives and mixtures thereof can be used as N-carboxy-anhydride monomer according to general structure III. Preferably L-amino acid derivatives are used for improved biodegradability.

Leucine derivatives, alanine derivatives, fenylalanine derivatives, phenylgrlycine derivatives, valine derivatives, isoleucine derivatives and methionine derivatives are particularly preferred as N-carboxy-anhydrides according to general structure III.

Typical N-carboxy-anhydrides according to structure III are given in Table 1 without being limited thereto.

TABLE 1

| | |
|---|---|
| 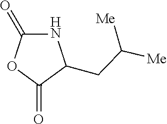 [SMILES: CC(C)CC1NC(=O)OC1=O] | NCA-1 |
| 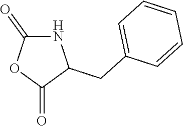 [SMILES: O=C1NC(Cc2ccccc2)C(=O)O1] | NCA-2 |
| 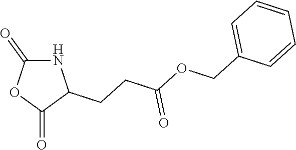 [SMILES: O=C(CCC1NC(=O)OC1=O)OCc1ccccc1] | NCA-3 |
| 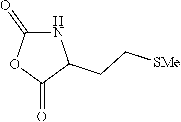 [SMILES: CSCCC1NC(=O)OC1=O] | NCA-4 |
| 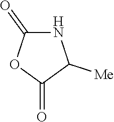 | NCA-5 |
| 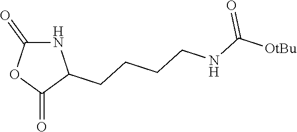 [SMILES: CC(C)(C)OC(=O)NCCCCC1NC(=O)OC1=O] | NCA-6 |
| 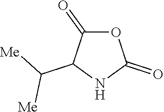 | NCA-7 |
| 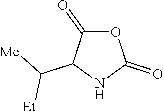 [SMILES: CCC(C)C1NC(=O)OC1=O] | NCA-8 |
| 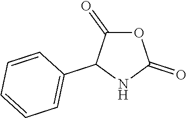 [SMILES: O=C1NC(c2ccccc2)C(=O)O1] | NCA-9 |

### A.1.2. N-Carboxy-Anhydride Monomer According to General Structure IV

In a preferred embodiment R₂ is selected from the group consisting of a hydrogen, a substituted or unsubstituted alkyl group, a substituted or unsubstituted aralkyl group and a substituted or unsubstituted aryl group.

In another preferred embodiment R₃ is selected from the group consisting of a substituted or unsubstituted alkyl group, a substituted or unsubstituted aralkyl group and a substituted or unsubstituted aryl group.

In a further preferred embodiment, R₂ and R₃ represent the necessary atoms to form a five or six membered ring.

Typical examples of N-carboxy-anhydrides according to general structure IV are given below without being limited thereto.

TABLE 2

| | |
|---|---|
| 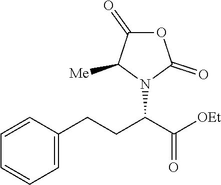 [SMILES: CCOC(=O)[C@H](CCc1ccccc1)N1C(=O)OC(=O)[C@@H]1C] | SNCA-1 |
| 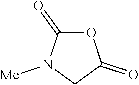 | SNCA-2 |
| 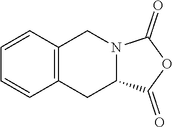 [SMILES: O=C1OC(=O)N2Cc3ccccc3C[C@@H]12] | SNCA-3 |
| 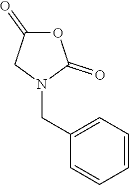 [SMILES: O=C1CN(Cc2ccccc2)C(=O)O1] | SNCA-4 |
| 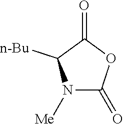 | SNCA-5 |
| 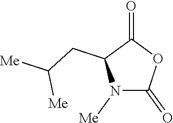 [SMILES: CC(C)C[C@H]1C(=O)OC(=O)N1C] | SNCA-6 |
| 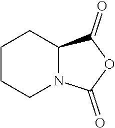 [SMILES: O=C1OC(=O)N2CCCC[C@@H]12] | SNCA-7 |
| 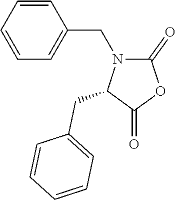 [SMILES: O=C1OC(=O)N(Cc2ccccc2)[C@H]1Cc1ccccc1] | SNCA-8 |
| 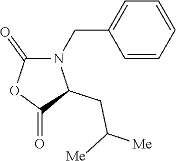 [SMILES: CC(C)C[C@H]1C(=O)OC(=O)N1Cc1ccccc1] | SNCA-9 |
| 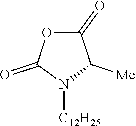 | SNCA-10 |

N-carboxy-anhydrides (NCA's) have been prepared using different synthetic methodologies, starting with the oldest method, known as Leuchs' method, starting from chloroformate acylation of the amino acid, followed by conversion to the corresponding NCA via its acid chloride. Several variants have been published on this methods, by Wessely and by Katchalski, respectively using a mixed anhydride method and a conversion using PBr₃. Probably, the most well-known method is the Fuchs-Farting method, using phosgene for direct conversion of the amino acid to the corresponding NCA. For safety reasons, phosgene has been replaced by di- or triphosgene in later research. Over the last years, several phosgene free methodologies have been disclosed. The methodologies have been reviewed by Secker et al. (Macromol. Biosci., 15, 881-891 (2015)).

### A.2. The Core

The core of the capsule according to the invention contains an organic compound. Preferably, the organic compound is a substantially low volatile compound. Substantially low volatile is defined as having a boiling point of at least 150° C. at 1013 mPas.

More preferably, the organic compound is a hydrophobic compound, meaning, having an octanol-water partition coefficient, expressed as log K_{ow} of at least 0.3. Without being bound by any theory, it is thought that a hydrophobic compound in the oleophilic drops during the interfacial polymerization keeps the formed poly(amino acid) chains having a hydrophilic character, to the outside of the drops resulting in a strong and dense sphere polymeric shell.

The average particle size of the capsules of the invention is preferably from 0.05 μm to 10 μm, more preferably from 0.07 μm to 5 μm and most preferably from 0.1 μm to 3 μm. Capsules according to the present invention having an average particle size below 1 μm are particularly preferred. Capsules having an average particle size below 1 μm are particularly useful for drug delivery and other pharmaceutical applications where the capsules have to be introduced in the animal or human body.

### B. The encapsulation Method

### B.1. Preparation Method of Capsules

The capsules according to the present invention are preferably prepared using a ring opening polymerization method, more preferably using interfacial ring opening polymerization.

The interfacial polymerization method according to the invention allows the preparation of capsules in a single step process and over a broad scope of functionalities and particle sizes, making it especially suitable for an industrial production process, more particularly for a continue industrial process. By simply adjusting the ratios of the monomers according to general structure III and general structure IV, the technology can easily be tuned towards the functionality to be encapsulated and the physical properties can easily be adjusted towards different applications without major changes in the process conditions leading to a robust technology with considerable latitude towards industrialization.

The ring opening polymerization of N-carboxy-anhydrides has been reviewed by Cheng and Deming (Top. Curr. Chem., 310, 1-26 (2012)). Primary and optionally secondary amines are the most obvious initiators and are widely used to initiate the ring opening polymerization via nucleophilic initiation. Basic initiators can initiate the ring opening polymerization via an activated monomer mechanism, starting by deprotonation of the NCA's followed by ring opening polymerization. When amine initiators are used, both mechanisms often run in parallel. Transition metal initiation is known to give better control on the polymerization. The use of hexamethyldisilazane as initiator has also been disclosed for better controlling the polymerization.

In a further preferred embodiment the ratio of an N-carboxy-anhydride according to general structure III on an N-carboxy-anhydride according to general structure IV is between 50 to 1 and 2 to 1, more preferably between 40 to 1 and 3 to 1 and most preferably between 25 to 1 and 5 to 1.

In a particularly preferred interfacial ring opening polymerization method for the preparation of the capsules according to the present invention, the N-carboxy-anhydride monomers according to general structure III and IV and the organic compound to be encapsulated are dissolved in a substantially water immiscible solvent and emulsified in an aqueous solution containing a polymerization initiator. Upon emulsifying and optionally removing said substantially water immiscible solvent, the ring opening polymerization is initiated at the interface. Upon propagation, a polypeptide shell is formed at the organic-water interface, generating a core-shell structure, encapsulating the organic compound. The obtained polymeric shell is mechanically strong and stable and allows the capsule to be isolated from the liquid wherein the capsules have been prepared.

If the organic compound is a liquid, dissolving in a substantially water immiscible solvent can be omitted and the NCA's can be directly dissolved in the liquid organic compound. The capsules according to the present invention are particularly suited to hold up liquid organic compounds in the core. Micellar based capsules are much less suited to encapsulate and hold up liquid core material. Indeed, the shell of a micellar system is in many cases too permeable with respect to a polymeric shell obtained by the encapsulation method of the invention.

A particularly preferred interfacial ring opening polymerization method comprises the steps of
a) dissolving a N-carboxy-anhydride monomer according to general structure III, a N-carboxy-anhydride monomer according to general structure IV and an organic compound in a water immiscible solvent; and
b) dissolving a polymerization initiator in an aqueous liquid; and
c) emulsifying the solution obtained in step a) into the aqueous liquid of step b); and
d) optionally evaporating the water immiscible solvent; and
e) polymerizing the N-carboxy-anhydride monomers according to general structure III and IV.

The particle size of the capsules of the invention is modified by modifying the emulsification technology, the use of an emulsification aid and the ratio of an emulsification aid to the shell and core during emulsification, the nature of the emulsification aid, changing the viscosity of the continuous or dispersed phase, the ratio of the continuous and dispersed phase, the nature of the core content and the nature of the shell monomers. High shear technologies and ultrasound based technologies are particularly preferred as emulsification technologies. The particle size of the capsules according to the present invention can be tuned by tuning the shear in high shear technologies or by changing the power and amplitude upon sonification.

### B.2. Initiators

Di- or multifunctional primary or secondary amines or mixtures thereof are particularly preferred initiators for the ring opening polymerization of the NCA's. The initiators are water soluble and can be functionalized with additional hydrophilic functional groups, preferably selected from the group consisting of a carboxylic acid or salt thereof, a sulfonic acid or salt thereof, a phosphonic acid or salt thereof, a phosphate ester or salt thereof, a sulfate ester or salt thereof, a poly-hydroxyl functionalized group, a poly(ethylene glycol), an ammonium group, a sulfonium group and a phosphonium group.

Typical initiators are given in Table 3 without being limited thereto

The incorporation of a poly(ethylene gycol) functional group is particularly useful to give stealth properties to the capsules of the invention if used as drug delivery system in the human or animal body. These stealth properties are required to avoid uptake by the reticuloendothelial system and only release drug at the required site in a controlled manner.

TABLE 3

| | |
|---|---|
| 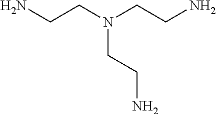 | INI-1 |
| 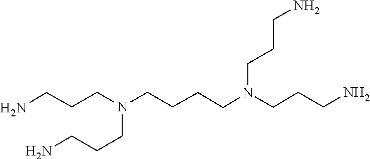 [SMILES: NCCCN(CCCN)CCCCN(CCCN)CCCN] | INI-2 |
| 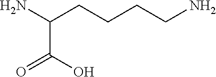 | INI-3 |
| 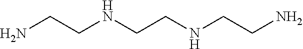 | INI-4 |
| 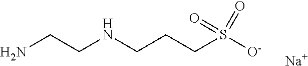 [SMILES: NCCNCCCS(=O)(=O)[O-] ; [Na+]] | INI-5 |
| 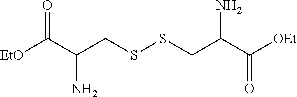 [SMILES: CCOC(=O)C(N)CSSCC(N)C(=O)OCC] | INI-6 |
| 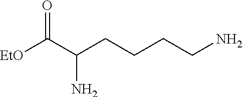 [SMILES: CCOC(=O)C(N)CCCCN] | INI-7 |
| 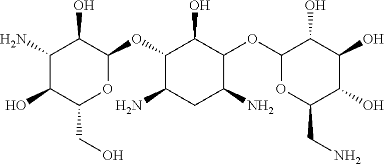 [SMILES: NC[C@H]1OC(OC2[C@@H](N)C[C@@H](N)[C@H](O[C@H]3O[C@H](CO)[C@@H](O)[C@H](N)[C@H]3O)[C@H]2O)[C@H](O)[C@@H](O)[C@@H]1O] | INI-8 |
| 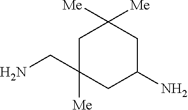 [SMILES: CC1(C)CC(N)CC(C)(CN)C1] | INI-9 |
| 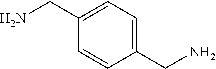 | INI-10 |
| 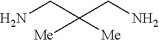 | INI-11 |
| 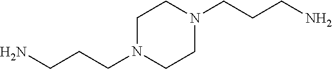 [SMILES: NCCCN1CCN(CCCN)CC1] | INI-12 |
| 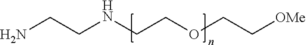 | INI-13 |
| 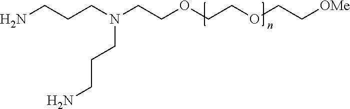 | INI-14 |

### B.3. Crosslinker

In a further preferred embodiment, the shell of the capsule further comprises a crosslinker. After biocompatibility and biodegradability, one of the most basic requirements of a capsule is stability in the medium wherein it has to function or has to be stored, e.g. the human body for a drug delivery system. If a system is not stable in its medium, this could result in a preliminary burst release of the payload or in non-targeted areas. Increased stability results in increased storage stability and for drug delivery systems, in an increased blood circulation time and increased bioavailability. With a crosslinker, the stability and mechanical resistance of the shell of the capsule can be modified to meet the specifications of the system in which the capsule is used. Further, the use of a crosslinker makes it possible to precisely control the drug release in the use of a drug delivery purpose of the capsules of the invention.

Any crosslinker known to crosslink amine functionalized polymers can be used. Preferred crosslinkers are selected from the group consisting of di- or multifunctional isocyanates, di- or multifunctional β-keto-esters, di- or multifunctional β-keto-amides, di- or multifunctional 1,3-diketones, di- or multifunctional epoxides or oxetanes, di- or multifunctional anhydrides, di- or multifunctional N-carboxy-anhydrides, di- or multifunctional Michael acceptors such as acrylates, methacrylates, maleimides, vinyl sulfones and the like and di- or multifunctional five membered carbonates.

### B.4. Emulsification Aid

Preferably, an additional emulsification aid is used during the emulsification step of the preparation of the capsule. Typical emulsification aids are selected from polymers and surfactants. The polymers and surfactants can be co-reactive polymers or surfactants, e.g. functionalized with primary and secondary amines, taking the role of both initiator and emulsification aid, leading to so called self-dispersing capsules. The surfactant can be anionic, non-ionic, cationic or zwitterionic. As stabilizing polymers, hydroxyl functionalized polymers are particularly preferred, preferably selected from polysaccharides and poly(vinyl alcohol) or poly(vinyl alcohol) copolymers or derivatives thereof.

### C. Fields of Application

The encapsulation technology, disclosed in the present invention is particularly useful in the field of personal care, pharmaceuticals, nutrition, agrochemicals and household applications, especially for controlling the release of the active components or protecting the active components from hydrolysis or oxidation. Examples are encapsulation of food ingredients, probiotics, fragrances and flavours, agrochemicals, flame retardants and last but not least, active pharmaceutical ingredients.

More general, the component in the core of the capsule preferably has an octanol-water partition coefficient, expressed as log K_{ow} of at least 0.3, more preferably of at least 0.5 and most preferably of at least 1.

The octanol-water partition coefficient is defined as follows,

$${K}_{ow} = {C}_{op} / {C}_{w}$$

where Cₒₚ and C_{w} are the concentrations of the compound under consideration in g L⁻¹ at 25° C., respectively in the octanol rich phase and the water rich phase.

The encapsulation technology according to the present invention is particularly of interest for the encapsulation of substantially non-reactive hydrophobic components such as marine oils, vegetable oils, and essential oils. The technology is also particularly of interest for the encapsulation of fragrances, flavors and insect repellents.

The encapsulation technology according to the present invention is further particularly of interest for the encapsulation of active pharmaceutical ingredients and agrochemicals.

More particularly, the encapsulation technology is useful in the encapsulation of active pharmaceutical ingredients such as an anti-cancer drug, a vaccine, a peptide, a protein, a sonosensitizer, a carrier for a drug, a gene, a growth factor such as recombinant bone morphogenetic protein (rhBMP-2), progesterone, procaine hydrochloride, bovine serum albumin, benzocaine, insulin, etc. The capsules of the invention are particularly suitable to be incorporated in a pharmaceutical composition for the treatment of cancer.

Capsules of the invention can be used in the treatment of cancer such as embolotherapy as disclosed in EP2891485A. These microspheres in an embolotherapy are used in a liquid when inserted into the human body, but are preferably maintained in a solid state for stable storage. In another aspect of the invention, the capsules of the invention are suitable in sonodynamic treatment of a metastatic disease, micrometastatic disease, or in the treatment of multiple primary tumours.

For use in any of the medical treatment methods described above, the capsules of the invention will generally be provided in a pharmaceutical composition together with at least one pharmaceutically acceptable carrier or excipient. Such pharmaceutical compositions may be formulated using techniques well known in the art. The route of administration will depend on the intended use. Typically, these will be administered systemically and may thus be provided in a form adapted for parenteral administration, e.g. by intradermal, subcutaneous, intraperitoneal or intravenous injection.

Suitable pharmaceutical compositions include suspensions and solutions which contain the capsules of the invention together with one or more inert carriers or excipients. Suitable carriers include saline, sterile water, phosphate buffered saline and mixtures thereof. The compositions may additionally include other agents such as emulsifiers, suspending agents, dispersing agents, solubilisers, stabilisers, buffering agents, wetting agents, preserving agents, etc. The pharmaceutical compositions may be sterilised by conventional sterilisation techniques. Solutions containing the particles may be stabilised, for example by the addition of agents such as viscosity modifiers, emulsifiers, solubilising agents, etc.

Preferably, the pharmaceutical compositions will be used in the form of an aqueous suspension or dispersion of the capsules in water or a saline solution, e.g, phosphate-buffered saline. The particles may be supplied in the form of a lyophilised powder for reconstitution at the point of use, e.g. for reconstitution in water, saline or phosphate-buffered saline.

The capsule according to the invention is particularly useful in a consumer product which is selected from the group consisting of a shampoo, a hair conditioner, a hair rinse, a hair refresher, a hair fixative or styling aid, a hair bleach, a hair dye or colorant, a soap, a body wash, a cosmetic preparation, an all-purpose cleaner, a bathroom cleaner, a floor cleaner, a window cleaner, a bath tissue, a paper towel, a disposable wipe, a diaper rash cream or balm, a baby powder, a diaper, a bib, a baby wipe, an oral care product, a tooth paste, an oral rinse, an tooth whitener, a denture adhesive, a chewing gum, a breath freshener, an orally dissolvable strips, a chewable candy, a hard candy, a hand sanitizer, an anti-inflammatory balm, an anti-inflammatory ointment, an anti-inflammatory spray, a health care device, a dental floss, a toothbrush, a tampon, a feminine napkin, a personal care product, a sunscreen lotion, a sunscreen spray, a wax-based deodorant, a glycol type deodorant, a soap type deodorant, a facial lotion, a body lotion, a hand lotion, a body powder, a shave cream, a bath soak, an exfoliating scrub, a foot cream, a facial tissue, a cleansing wipe, a fabric care product, a fabric softener, a fabric refresher, an ironing water, a liquid laundry detergent, a liquid dish detergent, an automatic dish detergent, a unit dose tablet or capsule, a scent booster, a drier sheet, a fine fragrance, a solid perfume, a powder foundation, a liquid foundation, an eye shadow, a lipstick or lip balm, an Eau De Toilette product, a deodorant, a rug deodorizer, a candle, a room deodorizer, a disinfectant, an anti-perspirant, an roll-on product, and an aerosol product.

### D. Examples

### D.1. Materials

All compounds are supplied by TCI Europe unless otherwise specified.
L-phenylalanine N-carboxy anhydride and D-phenylalanine N-carboxy anhydride can be prepared according to standard methods as disclosed by Gabashvill et al. (Journal of Physical Chemistry B, 111(38), 11105-11110 (2007)) and Otake et al. (Angewandte Chemie, International Edition, 57(35), 11389-11393 (2018)).
L-leucine N-carboxy anhydride and D-leucine N-carboxy anhydride can be prepared according to standard methods as disclosed by Baars et al. (Organic Process Research and Development, 7(4), 509-513 (2003)).
N-[1-(S)-Ethoxycarbonyl-3-phenylpropyl]-L-alanine-N-carboxyanhydride was supplied by TCI.
Mowiol 4 88 is a poly(vinyl alcohol) supplied by Kuraray.
Marlon A365 is an anionic surfactant supplied by Sasol Germany GMBH.
Tris(2-aminoethyl)amine was supplied by TCI.
Crosslinker-1 is a trifunctional β-keto-ester according to the following structure, which can be prepared as disclosed by Speisschaert et al. (Polymer, 172, 239-246 (2019)).

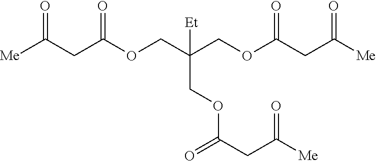 [SMILES: CCC(COC(=O)CC(C)=O)(COC(=O)CC(C)=O)COC(=O)CC(C)=O]

Caryofyllene is a hydrophobic organic compound and was supplied by Aldrich.
CATSURF-1 is a cationic surfactant according to the following structure, which can be prepared as disclosed in WO2018137993 (Agfa N.V) as Surf-3.

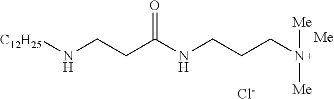

### D.2. Methods

### D.2.1. Particle Size Measurement

The particle size of the capsules was measured using a Zetasizer™ Nano-S (Malvern Instruments, Goffin Meyvis), which is based on Dynamic Light Scattering. The capsules are dispersed in deionized water and the measuring temperature is 23° C.

D.2.2. Thin Layer Chromatography—Mass Spectrometric analysis

A Thin Layer Chromatography (TLC) methodology was used for the analysis of the prepared N-carboxy-anhydrides.

A sample of the prepared N-carboxy anhydride was dissolved in ethyl acetate. An excess of indoline was added and the N-carboxy anhydride was allowed to react for 30 minutes at room temperature. The mixture was analyzed on a Grace Reveleris RP C18 TLC plate, using methanol/0.5 M NaCl 60/40 as eluent. The spots, detectable using a 254 nm UV lamp were analyzed by mass spectroscopy, according to following method: A TLC was run under circumstances given above. The TLC was analyzed using a CAMAG™ TLC-MS interface coupled to an AmaZon™ SL mass spectrometer (supplied by Brüker Daltonics) via an Agilent™ 1100 HPLC pump. First a blank spectrum was taken by eluting a spot on the TLC plate where no compounds are present with a 0.01 molar solution of ammonium acetate in methanol. A second spectrum of the compound to be analyzed was taken by eluting the spot of the compound under consideration with a 0.01 molar solution of ammonium acetate in methanol. The first spectrum was subtracted from the second spectrum, giving the spectrum of the compound to be analyzed.

### D.2.3. The Enzymatic Degradation

Aqueous dispersions were prepared containing 1 wt. % of comparative capsules or inventive capsules and 1 wt. % of the protease from Bacillus lichenoformis (Type VII, lyophilized, 7-15 units/mg, supplied by Merck) in water. These dispersions were incubated at 40° C.

After one week a sample of each dispersion was taken and analyzed using TLC in comparison with a 1 wt. % dispersion of each capsule, which was not incubated with the enzyme.

For TLC, a Grace Reveleris RP C18 TLC plate was used. MeOH/0.5 M NaCl was used as eluent and ninhydrine was used as detection method.

### D.3. Example 1

This example illustrates the improved biodegradability of the capsules according to the present invention.

### Preparation of (10aS)-10,10a-dihydro-5H-oxazolo[3,4-b]isoquinoline-1,3-dione (SNCA-3)

5 g (S)-(-)-1,2,3,4-tetrahydroisoquinoline-3-carboxylic acid was added to 100 ml tetrahydrofuran. The reaction vessel was coupled to a scrubber, containing a 1 N sodium hydroxide solution. 4.19 g triphosgene was added to the reaction mixture and the reaction was stirred for two hours at room temperature under an inert atmosphere. (S)-(-)-1,2,3,4-tetrahydroisoquinoline-3-carboxylic acid gradually dissolved. The reaction mixture was heated to 65° C. and the reaction was allowed to continue for 3 hours at 65° C. The reaction mixture was allowed to cool down to room temperature and the solvent was removed under reduced pressure. 100 ml n.-hexane was added and the mixture was treated with ultrasound to crystallize (10aS)-10,10a-dihydro-5H-oxazolo[3,4-b]isoquinoline-1,3-dione. (10aS)-10,10a-dihydro-5H-oxazolo[3,4-b]isoquinoline-1,3-dione only partially crystallized. The crystalline fraction was isolated and the residue was isolated by evaporating the solvent. The residue was redissolved in 5 ml tetrahydrofuran. The crystallized (10aS)-10,10a-dihydro-5H-oxazolo[3,4-b]isoquinoline-1,3-dione, dispersed in n·-hexane was added to the dissolved residue and the mixture was stirred for 15 minutes at room temperature. (10aS)-10,10a-dihydro-5H-oxazolo[3,4-b]isoquinoline-1,3-dione crystallized from the medium, was isolated by filtration and dried. 4.56 g (y: 80%) of (10aS) -10,10a-dihydro-5H-oxazolo[3,4-b]isoquinoline-1,3-dione was isolated.

(10aS)-10,10a-dihydro-5H-oxazolo[3,4-b]isoquinoline-1,3-dione was analyzed according to the TLC-method described in § D.2.2. A compound having a molecular mass of 278 g/mol was almost the only compound detectable in TLC, corresponding to the following structure.

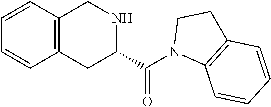 [SMILES: O=C([C@@H]1Cc2ccccc2CN1)N1CCc2ccccc21]

This clearly indicates that (10aS)-10,10a-dihydro-5H-oxazolo[3,4-b]isoquinoline-1,3-dione was formed to a very large extend.

### Preparation of Comparative Capsule Dispersion COMPCAP-1

A first solution was prepared by dissolving 1.5 g L-phenylalanine N-carboxy anhydride, 1.5 g L-leucine N-carboxy anhydride, 0.336 g crosslinker-1 and 3.46 g caryofyllene in 18 ml ethyl acetate.

A second solution was prepared by dissolving 0.692 g Mowiol 4 88, 0.389 g Marlon A365 and 0.127 g tris(2-aminoethyl)amine in 30 ml water.

The first solution was added to the second solution using mixing with an Ultra Turrax T25 (IKA) at 5000 rpm for 5 minutes while maintaining the temperature of the emulsion between 20 and 30° C. The ethyl acetate was removed under reduced pressure up to a weight of 30 g of the total dispersion and the polymerization was allowed to continue at room temperature for 24 hours.

The particle size distribution is from 1 μm to 5 μm.

### Preparation of Comparative Capsule Dispersion COMPCAP-2

A first solution was prepared by dissolving 0.75 g L-phenylalanine N-carboxy anhydride, 0.75 g D-phenylalanine N-carboxy anhydride, 0.75 g L-leucine N-carboxy anhydride, 0.75 g D-leucine N-carboxy anhydride, 0.336 g crosslinker-1 and 3.46 g caryofyllene in 18 ml ethyl acetate.

A second solution was prepared by dissolving 0.692 g Mowiol 4 88, 0.389 g Marlon A365 and 0.127 g tris(2-aminoethyl)amine in 30 ml water.

The first solution was added to the second solution using mixing with an Ultra Turrax T25 (IKA) at 5000 rpm for 5 minutes while maintaining the temperature of the emulsion between 20 and 30° C. The ethyl acetate was removed under reduced pressure up to a weight of 35 g of the total dispersion and the polymerization was allowed to continue at room temperature for 24 hours.

The particle size distribution is from 1 μm to 5 μm.

### Preparation of Inventive Capsule Dispersion INVCAP-1

A first solution was prepared by dissolving 1.25 g L-phenylalanine N-carboxy anhydride, 1.25 g L-leucine N-carboxy anhydride, 0.221 g N-[1-(S)-Ethoxycarbonyl-3-phenylpropyl]-L-alanine-N-carboxyanhydride, 0.294 g crosslinker-1 and 3.126 g caryofyllene in 18 ml ethyl acetate.

A second solution was prepared by dissolving 0.625 g Mowiol 4 88, 0.352 g Marlon A365 and 0.111 g tris(2-aminoethyl)amine in 30 ml water.

The first solution was added to the second solution using mixing with an Ultra Turrax T25 (IKA) at 5000 rpm for 5 minutes while maintaining the temperature of the emulsion between 20 and 30° C. The ethyl acetate was removed under reduced pressure up to a weight of 30 g of the total dispersion and the polymerization was allowed to continue at room temperature for 24 hours.

The particle size distribution is from 1 μm to 5 μm.

### Preparation of Inventive Capsule Dispersion INVCAP-2

A first solution was prepared by dissolving 1.25 g L-phenylalanine N-carboxy anhydride, 1.25 g L-leucine N-carboxy anhydride, 0.442 g N-[1-(S)-Ethoxycarbonyl-3-phenylpropyl]-L-alanine-N-carboxyanhydride, 0.308 g crosslinker-1 and 3.366 g Caryofyllene in 18 ml ethyl acetate.

A second solution was prepared by dissolving 0.673 g Mowiol 4 88, 0.379 g Marlon A365 and 0.117 g tris(2-aminoethyl)amine in 30 ml water.

The first solution was added to the second solution using mixing with an Ultra Turrax T25 (IKA) at 5000 rpm for 5 minutes while maintaining the temperature of the emulsion between 20 and 30° C. The ethyl acetate was removed under reduced pressure up to a weight of 30 g of the total dispersion and the polymerization was allowed to continue at room temperature for 24 hours.

The particle size distribution is from 1 μm to 5 μm.

### Preparation of Inventive Capsule Dispersion INVCAP-3

A first solution was prepared by dissolving 1.25 g L-phenylalanine N-carboxy anhydride, 1.25 g L-leucine N-carboxy anhydride, 0.885 g N-[1-(S)-Ethoxycarbonyl-3-phenylpropyl]-L-alanine-N-carboxyanhydride, 0.336 g crosslinker-1 and 3.848 g caryofyllene in 18 ml ethyl acetate.

A second solution was prepared by dissolving 0.770 g Mowiol 4 88, 0.433 g Marlon A365 and 0.127 g tris(2-aminoethyl)amine in 30 ml water.

The first solution was added to the second solution using mixing with an Ultra Turrax T25 (IKA) at 5000 rpm for 5 minutes while maintaining the temperature of the emulsion between 20 and 30° C. The ethyl acetate was removed under reduced pressure up to a weight of 30 g of the total dispersion and the polymerization was allowed to continue at room temperature for 24 hours.

The particle size distribution is from 1 μm to 5 μm.

### Preparation of Inventive Capsule Dispersion INVCAP-4

A first solution was prepared by dissolving 1.25 g L-phenylalanine N-carboxy anhydride, 1.25 g L-leucine N-carboxy anhydride, 0.147 g (10aS)-10,10a-dihydro-5H-oxazolo[3,4-b]isoquinoline-1,3-dione, 0.294 g crosslinker-1 and 3.052 g caryofyllene in 18 ml ethyl acetate.

A second solution was prepared by dissolving 0.610 g Mowiol 4 88, 0.344 g Marlon A365 and 0.111 g tris(2-aminoethyl)amine in 30 ml water.

The first solution was added to the second solution using mixing with an Ultra Turrax T25 (IKA) at 5000 rpm for 5 minutes while maintaining the temperature of the emulsion between 20 and 30° C. The ethyl acetate was removed under reduced pressure up to a weight of 30 g of the total dispersion and the polymerization was allowed to continue at room temperature for 24 hours.

The particle size distribution is from 1 μm to 5 μm.

### Results of Enzymatic Degradation of Capsule Dispersions

The enzymatic degradation of the comparative capsule dispersions COMPCAP-1 and COMPCAP-2 and the inventive capsules INVCAP-1 to INVCAP-4 were measured according to the method described in § D.2.3.

The TLC chromatograms of the comparative capsule dispersions COMPCAP-1 and COMPCAP-2 hardly showed any additional compound compared to the reference samples. Only minor amounts of degradation products were visual. The TLC chromatograms of all of the inventive capsule dispersions, showed several degradation products with a very pronounced presence of a compound at an Rf of 0.95.

In order to proof that this compound was formed by enzymatic degradation of the shell of the capsule, the TLC analysis was coupled to mass spectroscopy as described in § D.2.2.

The major degradation product identified in the chromatograms was a dipeptide (Rf=0.95) consisting of one leucine and one phenylalanine unit as amino acid. This dipeptide was clearly formed by enzymatic degradation of the shells of all inventive capsules. The dipeptide was hardly detectable in the degradation experiments with the comparative capsule dispersions COMPCAP-1 and COMPCAP-2.

The colloid chemical stability of both the inventive and the comparative capsule dispersions was evaluated using a microscopical analysis. From this analysis, it became clear that both comparative capsules COMPCAP-1 and COMPCAP-2 remained stable in dispersed form upon incubation with the protease from Bacillus lichenoformis, while all inventive capsules flocculated during incubation. This flocculation indicates an increased enzymatic activity for the inventive capsules due to the change in chemical composition of the shell, leading to colloid chemical instability.

1-15. (canceled)

16. A capsule consisting of a polymeric shell surrounding a core, the core comprises an organic compound, the polymeric shell comprises a polypeptide comprising a moiety according to general formula I and a moiety according to general formula II 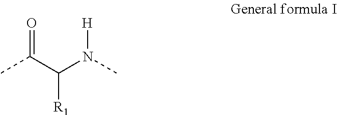
Wherein
R₁ is selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group. 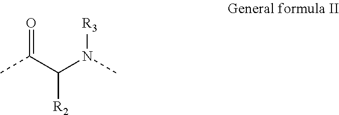
R₂ is selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₃ is selected from the group consisting of a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₂ and R₃ may represent the necessary atoms to form a five to eight membered ring.

17. The capsule according to claim 16 wherein the amount of the moiety according to general formula II is from 1 wt. % to 20 wt. % with respect to the total weight of the polymer shell.

18. The capsule according to claim 16 wherein the polymeric shell is obtainable by oligomerization or polymerization of a N-carboxy-anhydride monomer according to general structure III and a N-carboxy-anhydride monomer according to general structure IV. 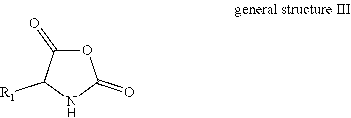
wherein
R₁ is selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group. 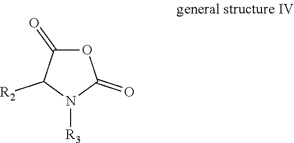
wherein
R₂ is selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₃ is selected from the group consisting of a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₂ and R₃ may represent the necessary atoms to form a five to eight membered ring.

19. The capsule according to claim 18 wherein the N-carboxy-anhydride monomer according to general structure III is selected from the group consisting of a glycine derivative, an alanine derivative, a leucine derivative, a phenylalanine derivative, a phenylglycine derivative, a valine derivative, a glutamic acid derivative, an aspartic acid derivative, a lysine derivative, an ornithine derivative, a histidine derivative, a methionine derivative, a cysteine derivative, an arginine derivative, a tryptophane derivative, a cysteine derivative, an isoleucine derivative, a tyrosine derivative and a serine derivative.

20. The capsule according to claim 18 wherein the ratio of the amount of N-carboxy-anhydride according to general structure III to the amount of N-carboxy-anhydride according to general structure IV is from 50 to 1 up to 2 to 1.

21. The capsule according to claim 16 wherein the organic compound has a octanol-water partition coefficient, expressed as log K_{ow} of 0.3 or more.

22. The capsule according to claim 18 wherein the organic compound has a octanol-water partition coefficient, expressed as log K_{ow} of 0.3 or more.

23. The capsule according to claim 16 wherein the organic compound is selected from the group consisting of marine oils, vegetable oils, essential oils, fragrances, flavours, insect repellents, flame retardants, active pharmaceutical ingredients and agrochemicals.

24. The capsule according to claim 18 wherein the organic compound is selected from the group consisting of marine oils, vegetable oils, essential oils, fragrances, flavours, insect repellents, flame retardants, active pharmaceutical ingredients and agrochemicals.

25. The capsule according to claim 16 further having an average particle size from 0.07 μm to 5 μm, the average particle size is measured by means of Dynamic Light Scattering of the capsules dispersed in deionized water at 23° C.

26. The capsule according to claim 16 wherein the polymeric shell comprises a crosslinker.

27. The capsule according to claim 18 wherein the polymeric shell comprises a crosslinker.

28. The capsule according to claim 16 wherein the polymeric shell comprises a dispersing group selected from the group consisting of a carboxylic acid or a salt thereof, a sulfonic acid or salt thereof, a phosphoric acid ester or a salt thereof, a phosphonic acid or salt thereof a, protonated amine, a protonated nitrogen containing heteroaromatic compound, a quaternized tertiary amine, a N-quaternized heteroaromatic group, a sulfonium and a phosphonium.

29. The capsule according to claim 16 wherein the organic compound is selected from the group consisting of an anti-cancer drug, a vaccine, a peptide, a protein and a sonosensitizer.

30. The capsule according to claim 18 wherein the organic compound is selected from the group consisting of an anti-cancer drug, a vaccine, a peptide, a protein and a sonosensitizer

31. A pharmaceutical composition comprising the capsule as claimed in claim 29 and a pharmaceutical carrier or excipient.

32. A consumer product comprising the capsule as defined in claim 16 wherein the consumer product is selected from the group consisting of a shampoo, a hair conditioner, a hair rinse, a hair refresher, a hair fixative or styling aid, a hair bleach, a hair dye or colorant, a soap, a body wash, a cosmetic preparation, an all-purpose cleaner, a bathroom cleaner, a floor cleaner, a window cleaner, a bath tissue, a paper towel, a disposable wipe, a diaper rash cream or balm, a baby powder, a diaper, a bib, a baby wipe, an oral care product, a tooth paste, an oral rinse, an tooth whitener, a denture adhesive, a chewing gum, a breath freshener, an orally dissolvable strips, a chewable candy, a hard candy, a hand sanitizer, an anti-inflammatory balm, an anti-inflammatory ointment, an anti-inflammatory spray, a health care device, a dental floss, a toothbrush, a tampon, a feminine napkin, a personal care product, a sunscreen lotion, a sunscreen spray, a wax-based deodorant, a glycol type deodorant, a soap type deodorant, a facial lotion, a body lotion, a hand lotion, a body powder, a shave cream, a bath soak, an exfoliating scrub, a foot cream, a facial tissue, a cleansing wipe, a fabric care product, a fabric softener, a fabric refresher, an ironing water, a liquid laundry detergent, a liquid dish detergent, an automatic dish detergent, a unit dose tablet or capsule, a scent booster, a drier sheet, a fine fragrance, a solid perfume, a powder foundation, a liquid foundation, an eye shadow, a lipstick or lip balm, an Eau De Toilette product, a deodorant, a rug deodorizer, a candle, a room deodorizer, a disinfectant, an anti-perspirant, an roll-on product, and an aerosol product.

33. A method of preparing the capsules as defined in claim 16 comprising the steps of:
a) dissolving a N-carboxy-anhydride monomer according to general structure III, a N-carboxy-anhydride monomer according to general structure IV and an organic compound in a water immiscible solvent: and
b) dissolving a polymerization initiator in an aqueous liquid: and
c) emulsifying the solution obtained in step a) into the aqueous liquid of step b); and
d) optionally evaporating the water immiscible solvent; and
e) polymerizing the N-carboxy-anhydride monomers according to general structure III and IV.

34. The method of preparing the capsules according to claim 33 wherein the polymerization initiator is a di- or multifunctional primary or secondary amine.
